(19) United States
(12) Patent Application Publication
(10) Pub. No.: US 2010/0275919 A1
(43) Pub. Date: Nov. 4, 2010

(54) RESPIRATORY SYSTEM AND RESPIRATORY MASK DEVICE

(75) Inventor: Ching-Lung Sung, Taichung City (TW)

Correspondence Address: SNYDER, CLARK, LESCH & CHUNG, LLP, 950 Herndon Parkway, Suite 365, HERNDON, VA, 20170, US

(73) Assignee: TOP VISION MEDICAL EQUIPMENT CONSULTANT CO., LTD., The Valley (AI)

(21) Appl. No.: 12/768,771

(22) Filed: Apr. 28, 2010

(30) Foreign Application Priority Data
May 1, 2009 (TW) 098114627

(51) Int. Cl. A61M 16/06; A61M 16/10

(52) U.S. Cl. 128/204.22; 128/206.21

(57) ABSTRACT

A respiratory system includes: a mask body adapted for attachment to a user's face; a gas supplier connected to the mask body to supply a gas to the mask body; a central control unit associated with the gas supplier to control temperature and humidity of the gas supplied from the gas supplier to the mask body; a sensor unit disposed in the mask body to produce sensing signals and to convert the sensing signals into a signal output that is related to at least temperature and humidity of the gas; and a transmitting unit to transmit the signal output to the central control unit. The temperature and humidity of the gas supplied from the gas supplier is controlled based on the signal output.

### CROSS-REFERENCE TO RELATED APPLICATION

This application claims priority of Taiwanese application no. 098114627, filed on May 1, 2009.

### BACKGROUND OF THE INVENTION

1. Field of the Invention

This invention relates to a respiratory system and a respiratory mask device, more particularly to a respiratory system and a respiratory mask device adapted to sense and adjust the humidity and temperature of gas supplied thereby.

2. Description of the Related Art

A conventional respiratory system is normally provided with a gas supplier to constantly and rapidly provide a gas to a mask device attached to the face of a wearer. The gas supplier includes an air blower or a turbo-compressor to create a negative pressure to guide the ambient air thereto and to create a positive pressure to guide the air to the mask device. A known respiratory system is further provided with a humidification chamber for adjusting the temperature and humidity of the ambient air introduced thereto and for supplying the gas with the predetermined temperature and humidity to the wearer of the mask device.

However, since the temperature and humidity of the environment will change due to factors, such as operation of an air conditioner and the current climate, and since the gas at the user end in the conventional respiratory system is manually adjusted, the gas supplied to the user is in an unstable state especially when a tube for supplying the gas from the conventional respiratory system to the mask device is relatively long. This is undesirable for users in need of long-term care.

### SUMMARY OF THE INVENTION

Therefore, an object of the present invention is to provide a respiratory system and a respiratory mask device that can overcome the aforesaid drawbacks associated with the prior art.

According to one aspect of this invention, a respiratory system is provided. The respiratory system comprises: a mask body adapted for attachment to a user's face; a gas supplier connected to the mask body to supply a gas to the mask body; a central control unit associated with the gas supplier to control temperature and humidity of the gas supplied from the gas supplier to the mask body; a sensor unit disposed in the mask body to produce sensing signals and to convert the sensing signals into a signal output that is related to at least temperature and humidity of the gas; and a transmitting unit to transmit the signal output to the central control unit, wherein the temperature and humidity of the gas supplied from the gas supplier is controlled based on the signal output.

According to another aspect of this invention, a respiratory mask device adapted for connection with a gas supplier, that has a central control unit to control temperature and humidity of a gas supplied to the respiratory mask device, is provided. The respiratory mask device comprises: a mask body adapted to be connected fluidly to the gas supplier; a sensor unit disposed in the mask body to produce sensing signals and to convert the sensing signals into a signal output that is related to at least temperature and humidity of the gas; and a transmitting unit adapted for electrical connection with the central control unit for transmitting the signal output to the central control unit.

### BRIEF DESCRIPTION OF THE DRAWINGS

Other features and advantages of the present invention will become apparent in the following detailed description of the preferred embodiments of the invention, with reference to the accompanying drawings, in which:

FIG. 1 is a perspective view of the preferred embodiment of a respiratory mask device according to the present invention;

FIG. 2 is a schematic diagram to illustrate the gas temperature and humidity control mechanism of a respiratory system according to the first embodiment of the present invention; and

FIG. 3 is a schematic diagram to illustrate the temperature and humidity control mechanism of a respiratory system according to the second embodiment of the present invention.

### DETAILED DESCRIPTION OF THE PREFERRED EMBODIMENTS

Before the present invention is described in greater detail with reference to the accompanying preferred embodiments, it should be noted herein that like elements are denoted by the same reference numerals throughout the disclosure.

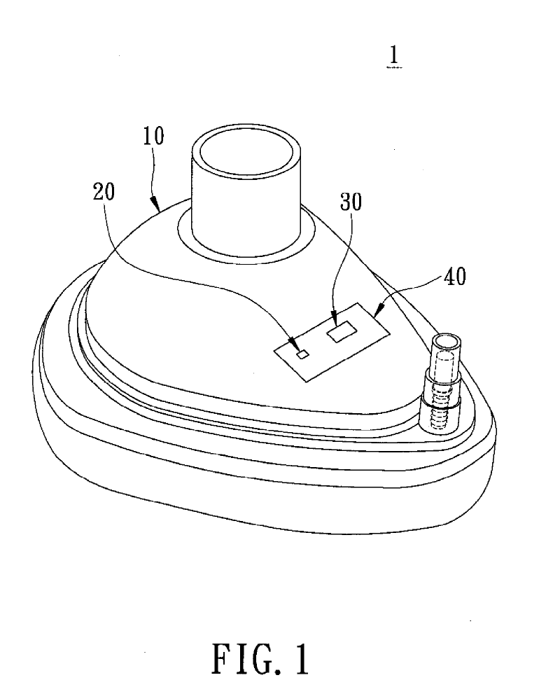

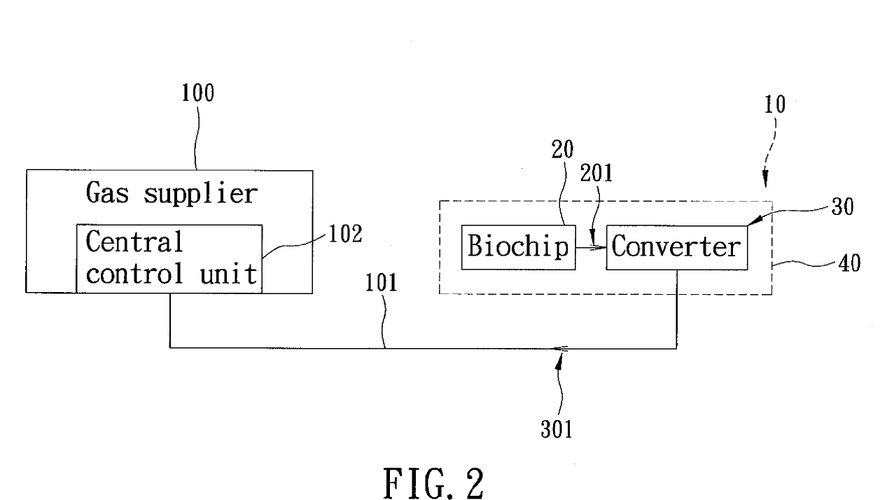

Referring to FIGS. 1 and 2, a respiratory system according to the first embodiment of the present invention is shown to include a respiratory mask device 1, and a gas supplier 100.

The respiratory mask device 1 includes a mask body 10 and a sensor unit disposed in the mask body 10.

The mask body 10 is adapted for attachment to a user's face and is to be connected to and for fluid communication with the gas supplier 100.

The sensor unit produces sensing signals 201 and converts the sensing signals 201 into a signal output 301 that is related to at least temperature and humidity of a gas flowing therethrough.

In this preferred embodiment, the sensor unit includes a biochip 20 and a converter 30. The biochip 20 is mounted on a printed circuit board 40 mounted on the mask body 10, and is configured to sense a gas condition inside the mask body 10 and to produce the sensing signals 201 including a temperature signal, a relative humidity signal, a pressure signal, a flow rate signal, an oxygen level signal and a carbon dioxide level signal. The converter 30 is also mounted on the printed circuit board 40, and is a single chip for receiving the sensing signals 201 and converting the sensing signals 201 into the signal output 301 that is related to information (i.e., the temperature, the relative humidity, the pressure, the flow rate, the oxygen level, and the carbon dioxide level inside the mask body 10) for computing temperature and humidity of the gas inside the mask body 10.

The gas supplier 100 is connected to the mask body 10 to supply the gas to the mask body 10 and includes a central control unit 102.

The respiratory mask device 1 further includes a transmitting unit that is for electrical connection with the converter 30 of the sensor unit and the central control unit 102 of the gas supplier 100 and that is used to transmit the signal output 301 to the central control unit 102. In this preferred embodiment, the transmitting unit includes a bus 101 connected to the converter 30 and the central control unit 102 of the gas supplier 100 for transmitting the signal output 301. The bus 101 in this preferred embodiment is also used for supplying power to the biochip 20 and the converter 30.

The central control unit 102 is used to control temperature and humidity of the gas supplied from the gas supplier 100 to the mask body 10 based on the signal output 301. Specifically, after the central control unit 102 receives the signal output 301 and computes the data concerning the temperature and humidity of the gas inside the mask body 10, it will give instructions to adjust the temperature and humidity of the gas to be supplied from the gas supplier 100 automatically and in real-time.

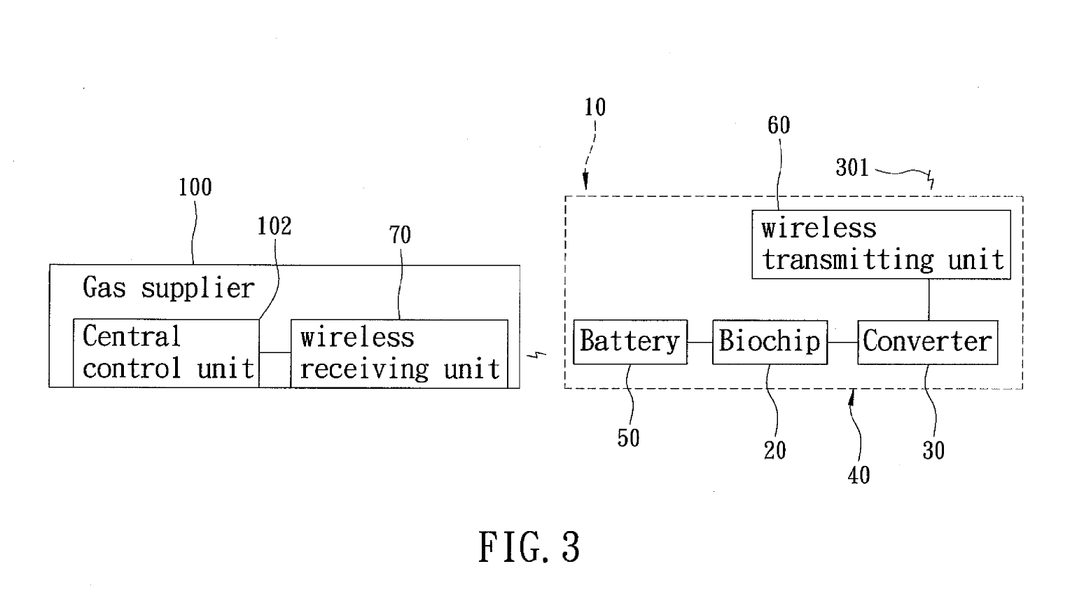

FIG. 3 shows a schematic diagram to illustrate the temperature and humidity control mechanism of a respiratory system according to the second embodiment of the present invention.

The second embodiment differs from the first embodiment only in that the mask body 10 further includes a rechargeable battery 50 that is mounted on the printed circuit board 40 and connected to the biochip 20 and the converter 30, that the bus 101 is replaced with a wireless transmitting unit 60 connected to the converter 30, and that the gas supplier 100 further includes a wireless receiving unit 70 connected to the central control unit 102.

By way of this configuration, the rechargeable battery 50 can serve as a power supply for the biochip 20 and the converter 30, and the signal output 301 can be transmitted wirelessly from the wireless transmitting unit 60 connected to the converter 30 of the mask body 10 to the wireless receiving unit 70 connected to the central control unit 102 of the gas supplier 100.

While the present invention has been described in connection with what are considered the most practical and preferred embodiments, it is understood that this invention is not limited to the disclosed embodiments but is intended to cover various arrangements included within the spirit and scope of the broadest interpretations and equivalent arrangements.

What is claimed is:

1. A respiratory system comprising:
a mask body adapted for attachment to a user's face;
a gas supplier connected to said mask body to supply a gas to said mask body;
a central control unit associated with said gas supplier to control temperature and humidity of the gas supplied from said gas supplier to said mask body;
a sensor unit disposed in said mask body to produce sensing signals and to convert said sensing signals into a signal output that is related to at least temperature and humidity of the gas; and
a transmitting unit to transmit said signal output to said central control unit, wherein the temperature and humidity of the gas supplied from said gas supplier is controlled based on said signal output.

2. The respiratory system of claim 1, wherein said sensing signals include a temperature signal, a humidity signal, a pressure signal, a flow rate signal, an oxygen level signal and a carbon dioxide level signal.

3. The respiratory system of claim 2, wherein said sensor unit includes a biochip configured to produce said sensing signals, and a converter to convert said sensing signals into said signal output.

4. The respiratory system of claim 3, wherein said transmitting unit includes a bus connected to said converter and said central control unit.

5. The respiratory system of claim 3, wherein said transmitting unit includes a wireless transmitting unit.

6. The respiratory system of claim 1, further comprising
a printed circuit board mounted on said mask body, said sensor unit being mounted on said printed circuit board.

7. The respiratory system of claim 6, further comprising
a battery mounted on said printed circuit board and connected to said sensor unit.

8. A respiratory mask device adapted for connection with a gas supplier that has a central control unit to control temperature and humidity of a gas supplied to said respiratory mask device, comprising:
a mask body adapted to be connected fluidly to the gas supplier;
a sensor unit disposed in said mask body to produce sensing signals and to convert said sensing signals into a signal output that is related to at least temperature and humidity of the gas; and
a transmitting unit adapted for electrical connection with the central control unit for transmitting said signal output to the central control unit.

9. The respiratory mask device of claim 8, wherein said sensing signals include a temperature signal, a humidity signal, a pressure signal, a flow rate signal, an oxygen level signal and a carbon dioxide level signal.

10. The respiratory mask device of claim 9, wherein said sensor unit includes a biochip configured to produce said sensing signals, and a converter to convert said sensing signals into said signal output.

11. The respiratory mask device of claim 10, wherein said transmitting unit includes a bus connected to said converter.

12. The respiratory mask device of claim 10, wherein said transmitting unit includes a wireless transmitting unit connected to said converter.

13. The respiratory mask device of claim 8, further comprising a printed circuit board mounted on said mask body, said sensor unit being mounted on said printed circuit board.

14. The respiratory mask device of claim 13, further comprising a battery mounted on said printed circuit board and connected to said sensor unit.
